(19) United States
(12) Patent Application Publication
(10) Pub. No.: US 2024/0232453 A9
(43) Pub. Date: Jul. 11, 2024

(54) METHOD FOR PREDICTING THE PERFORMANCE OF NOVEL INTERMETALLICS

(71) Applicant: Honeywell Federal Manufacturing & Technologies, LLC, Kansas City, MO (US)

(72) Inventors: Wesley Alexander Everhart, Overland Park, KS (US); Joseph Newkirk, Rolla, MO (US)

(21) Appl. No.: 18/492,210

(22) Filed: Oct. 23, 2023

Related U.S. Application Data
(60) Provisional application No. 63/418,372, filed on Oct. 21, 2022.

(51) Int. Cl. G06F 30/17; C21D 1/74; C21D 6/00; C22C 1/03; C22C 19/07; C22C 33/06; C22C 38/10; C22C 38/12; C22F 1/02; C22F 1/10; G06N 5/022; G16C 60/00

(52) U.S. Cl. CPC G06F 30/17; C21D 1/74; C21D 6/007; C22C 1/03; C22C 19/07; C22C 33/06; C22C 38/10; C22C 38/12; C22F 1/02; C22F 1/10; G06N 5/022; G16C 60/00

(57) ABSTRACT

Methods of modeling metal alloys and forming those alloys are provided. The method involves comparing the strain accommodation and cleavage energies of a base alloy comprising a first metal and a chemical element different from the first metal. If a predetermined difference between those energies would be achieved, the base alloy will be sufficiently ductile. If that predetermined difference would not be achieved, the base alloy will not be sufficiently ductile, and the base alloy is modified (e.g., by adding a ductility component) until the predetermined difference in energies would be achieved, at which point, the alloy can be formed using conventional methods or further modified to achieve the desired degree of ductility.

### CROSS-REFERENCE TO RELATED APPLICATIONS

The present application claims the priority benefit of U.S. Provisional Patent Application Ser. No. 63/418,372, filed Oct. 21, 2022, entitled METHOD FOR PREDICTING THE PERFORMANCE OF NOVEL INTERMETALLICS, the entirety of which is incorporated by reference herein.

### FEDERALLY SPONSORED RESEARCH OR DEVELOPMENT

This invention was made with Government support under Contract No. DE-NA-0002839, awarded by the United States Department of Energy/National Nuclear Security Administration. The Government has certain rights in the invention.

### BACKGROUND

### Field

The present disclosure relates to methods of modifying alloys to have improved ductilities compared to those of the starting (i.e., unmodified) alloys.

### Description of Related Art

With the first addition of vanadium to near equiatomic iron-cobalt alloys in 1932, the commercial use of these alloys has skyrocketed due to the significant amount of ductility enabled after quenching from the gamma phase. An additional benefit of the vanadium additions is an increase in ductility in the ordered state accompanied by a shift from intergranular fracture to transgranular fracture. The reported magnitudes of this increase range from 2.5% to 9.2% elongation, and the reason for the increase is still not well understood.

The wide range of reported values is possibly related to chemistry differences in the various works, the specific chemistry of many of these alloys relative to stoichiometry is often not well controlled or reported, and the thermal history of the materials isn't always clear or consistent. Very little work has been done to evaluate the effects of stoichiometry in Fe—Co alloys and the impact of stoichiometric defects on ductility.

A more critical look at how chemistry impacts ductility in Fe—Co and other alloys is needed. In general, the accommodation of strain of inorganic materials is governed by the motion of dislocations and their ability to cross-slip. The common von Mises criterion states that five independent slip planes are necessary for a material to be considered ductile. The stable magnetic structure of equiatomic Fe—Co is B2, an intermetallic, or ordered, form of the common BCC structure. In intermetallics, dislocations tend to form as a pair of dislocations separated by an anti-phase boundary (“APB”), often called a superdislocation, and thus the cross-slip of dislocations is complex. In B2 alloys, such cross-slip is most likely to occur between the {110} and {112} planes. Paidar et al. (Cross-slip of Superpartial Dislocations in Iron Aluminides, Philos. Mag. A Phys. Condens. Matter, Struct. Defects Mech. Prop. 81, 1065-1077 (2001)) derived an equation for the shear stress necessary to cross-slip from the {110} to the {112} plane as a function of the relative anti-phase boundary (APB) energies and the portion of the superdislocation that has cross-slipped. However, no work has been done to include changes in APB energies or cross-slip in the analysis of the observed ductility increase in Fe—Co alloys with the addition of vanadium.

With the connection between anti-phase boundary energies and the ductility of intermetallics, some efforts have been made to calculate or predict these APB energies through various methods, but a fairly wide range of values have been reported, and no actual measurements of APB energy in Fe—Co have been reported. Additionally, no theories have been presented that identify the impact of changes in APB energies or cross-slip on the observed ductility increase with the addition of vanadium.

There is a distinct need to develop a fundamental understanding of what drives ductility in Fe—Co and other alloys to enable informed development of specification limits and thermo-mechanical processing for improved mechanical performance. Such improvements could lead to better use in a variety of applications such as solenoids, electric vehicles, and magnetic shielding.

### SUMMARY

The present disclosure is broadly concerned with a method of forming a final alloy from a base alloy comprising a first quantity of a first metal and a first quantity of a chemical element different from the first metal. The method comprises determining whether a predetermined difference between the strain accommodation energy of the base alloy and the cleavage energy of the base alloy would exist. If the predetermined difference would exist, the final alloy is formed by subjecting the respective first quantities to an alloy formation process. If the predetermined difference would not exist, it is determined whether a modified base alloy would achieve the predetermined difference, with the modified base alloy comprising a modification chosen from:
(a) a second quantity of the first metal different from the first quantity of the first metal;
(b) a second quantity of the chemical element different from the first quantity of the chemical element;
(c) a ductility component different from the first metal and different from the chemical element, wherein ductility component would either be absent from the base alloy or would be present in the base alloy in a quantity different from that in the modified base alloy;
(d) two of (a), (b), or (c); or
(e) each of (a), (b), and (c).
   If the modified base alloy would achieve the predetermined difference, the final alloy is formed by subjecting the first metal, the chemical element, and any ductility component to an alloy formation process. If the modified base alloy would not achieve the predetermined difference, the modification process is repeated with one or more further modified base alloys until a further modified base alloy is identified that would achieve the predetermined difference, with the further modified base alloy having respective final quantities of the first metal, the chemical element, and any ductility component that may be present. The final alloy is then formed by subjecting the respective final quantities to an alloy formation process.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the transgranular cleavage energies for various crystallographic planes in equiatomic Fe—Co;

FIG. 2 is a graph of the energetic competition between strain and cleavage;

FIG. 3 is a graph showing APB energy extension from standard PAW potentials to APB results using PAW_pv and PAW_sv potentials;

FIG. 4 shows (a) vacancy, anti-site, and vanadium substitution point defect energies in Fe—Co, and (b) relative stabilities of anti-site and vanadium substitutional defects when they appear at APBs or GBs;

FIG. 5 provides (a) anti-phase boundary energies for various chemical defects and (b) representative pair correlation functions for vanadium-free and vanadium-containing alloys along {110} APBs;

FIG. 6 is a graph of cross-slip energies from the {110} to {112} plane;

FIG. 7 provides cleavage energies for various defects on {310} GBs and {110} crystal planes;

FIG. 8 is a ternary plot for the cleavage transition criteria as a function of chemistry, with the line on the figure denoting the transition between intergranular and transgranular cleavage;

FIG. 9 is a graph of representative disk bend test results for alloys with vanadium (2 at. % V, Co-lean) and without vanadium (equiatomic Fe—Co);

FIG. 10 illustrates the predicted change in strain to failure compared to the measured disk bend displacement for each alloy;

FIG. 11 provides scanning electron microscope (“SEM”) images of the fracture surface of (a) equiatomic Fe—Co and (b) 2 at. % V, Co-lean Fe—Co;

FIG. 12 contains graphs showing (a) predicted cleavage energies compared to the measured disk bend displacement for each alloy and (b) predicted cross-slip energy compared to the measured disk bend displacement for each alloy.

FIG. 13 is a graph depicting predicted change in strain to failure for corrected B2 chemistries compared to the measured disk bend failure displacement for each alloy;

FIG. 14 is a graph of (a) cross-slip energies and (b) predicted cleavage energies, each compared to the measured disk bend displacement for each updated alloy;

FIG. 15 is an inverse pole figure of the cracked region of a broken disk bend sample from (a) the equiatomic Fe—Co alloy and from (b) the 2 at. % vanadium cobalt-rich Fe—Co alloy;

FIG. 16 is a dislocation density map of the cracked region of a broken disk bend sample from (a) the equiatomic Fe—Co alloy and from (b) the 2 at. % vanadium cobalt-rich Fe—Co alloy; and

FIG. 17 is a dislocation density map of the cracked region of a broken disk bend sample from the 2 at. % cobalt-rich Fe—Co alloy.

### DETAILED DESCRIPTION

The present disclosure is broadly concerned with methods of altering the ductility of an alloy by predicting the impact on ductility of different quantities of one or more metals in the alloy and/or the introduction of an entirely new metal into the alloy.

### 1. Base Alloy

As used herein, “alloy” refers to a mixture of two or more chemical elements, at least one of which is a metal.

A “base alloy” refers to an alloy used as the starting alloy when following the methods described herein. For example, a “base alloy” could be an existing alloy whose ductility will be modeled, and possibly altered, as described below. A “base alloy” could also include an alloy that has not previously existed but whose ductility will be modeled (i.e., a hypothetical base alloy), and possibly altered, as described below.

Regardless, the base alloy will comprise a first metal and a chemical element different from the first metal.

First metals that could be present in the base alloy include any metal (alkali metals, alkaline earth metals, transition metals). Examples of suitable first metals include one or more of iron, cobalt, lithium, sodium, potassium, rubidium, cesium, francium, beryllium, magnesium, calcium, strontium, barium, radium, scandium, yttrium, titanium, zirconium, hafnium, rutherfordium, vanadium, niobium, tantalum, dubnium, chromium, molybdenum, tungsten, seaborgium, manganese, technetium, rhenium, bohrium, ruthenium, osmium, hassium, rhodium, iridium, meitnerium, nickel, palladium, platinum, darmstadtium, copper, silver, gold, roentgenium, zinc, cadmium, mercury, copernicium, aluminum, gallium, indium, thallium, nihonium, tin, lead, flerovium, bismuth, moscovium, polonium, livermorium, tennessine, or mixtures thereof.

Suitable chemical elements that are different from the first metal include metals (alkali metals, alkaline earth metals, transition metals), metalloids, interstitial elements, or mixtures thereof. Examples include one or more elements chosen from iron, cobalt, lithium, sodium, potassium, rubidium, cesium, francium, beryllium, magnesium, calcium, strontium, barium, radium, scandium, yttrium, titanium, zirconium, hafnium, rutherfordium, vanadium, niobium, tantalum, dubnium, chromium, molybdenum, tungsten, seaborgium, manganese, technetium, rhenium, bohrium, ruthenium, osmium, hassium, rhodium, iridium, meitnerium, nickel, palladium, platinum, darmstadtium, copper, silver, gold, roentgenium, zinc, cadmium, mercury, copernicium, aluminum, gallium, indium, thallium, nihonium, tin, lead, flerovium, bismuth, moscovium, polonium, livermorium, tennessine, boron, silicon, germanium, arsenic, antimony, tellurium, astatine, carbon, oxygen, sulfur, phosphorus, or mixtures thereof. In some embodiments, the chemical element is also a metal, but a metal that is different from the first metal.

In some embodiments, the base alloy might include one or more components in addition to the first metal and the chemical element different from the first metal (“additional components”). Such additional component could be any element other than noble gases as well as mixtures of elements other than noble gases, such as metals (alkali metals, alkaline earth metals, transition metals), metalloids, interstitial elements, or mixtures thereof. Examples of additional components include one or more of cobalt, lithium, sodium, potassium, rubidium, cesium, francium, beryllium, magnesium, calcium, strontium, barium, radium, scandium, yttrium, titanium, zirconium, hafnium, rutherfordium, vanadium, niobium, tantalum, dubnium, chromium, molybdenum, tungsten, seaborgium, manganese, technetium, rhenium, bohrium, ruthenium, osmium, hassium, rhodium, iridium, meitnerium, nickel, palladium, platinum, darmstadtium, copper, silver, gold, roentgenium, zinc, cadmium, mercury, copernicium, aluminum, gallium, indium, thallium, nihonium, tin, lead, flerovium, bismuth, moscovium, polonium, livermorium, tennessine, boron, silicon, germanium, arsenic, antimony, tellurium, astatine, carbon, oxygen, sulfur, phosphorus, or mixtures thereof.

In instances where the base alloy contains three or more total components (i.e., the first metal, the chemical element, and the additional component(s)), the first metal and the chemical element will generally be the two that are present in the base alloy at the two highest atomic percentages. Thus, in embodiments where the base alloy includes one or more additional components, it is preferred that the total quantity (atomic % and/or weight %) of the one or more additional components is less than the total quantity (atomic % and/or weight %) of the first metal. Additionally or alternatively, in embodiments where the base alloy includes one or more additional components, it is preferred that the total quantity (atomic % or weight %) of the one or more additional components is less than the total quantity (atomic % and/or weight %) of the chemical element different from the first metal.

One preferred base alloy comprises iron and cobalt. Another preferred base alloy comprises iron, cobalt, and vanadium.

The respective quantities of the first metal and the chemical element different from the first metal are not generally limited and can be selected as desired by one skilled in the art. However, in some embodiments, it is preferred that the chemical element different from the first metal is present in the alloy in a quantity that is within about 20 at. %, preferably within about 15 at. %, more preferably within about 10 at. %, even more preferably within about 5 at. %, and most preferably within about 2 at. % of the quantity of the first metal.

In one or more embodiments, the base alloy is stoichiometric or near-stoichiometric. In some embodiments, the base alloy is equiatomic or near-equiatomic. Additionally or alternatively, in some embodiments, the base alloy is an intermetallic. In one or more embodiments, the atomic ratio of the first metal to the chemical element different from the first element in the base alloy is about 0.8 to about 1.2, preferably about 0.9 to about 1.1, more preferably about 0.95 to about 1.05, and even more preferably about 1:1.

### 2. Methods of Forming Alloys

The method of forming alloys involves comparing the strain accommodation energy of the base alloy to the cleavage energy of the base alloy to determine if a predetermined or desired difference in those energies exist, meaning that the base alloy would be sufficiently ductile. It will be appreciated that this predetermined difference can be identified by one of ordinary skill in the art based on the degree of ductility desired in the alloy to be formed.

Strain accommodation energy and cleavage energy can be determined as described in Example 1.

a. Base Alloy Not Sufficiently Ductile

If the base alloy is not sufficiently ductile (i.e., the desired predetermined difference would not be achieved), the strain accommodation energy of a modified base alloy (I) can be compared to the cleavage energy of the modified base alloy (I). The modified base alloy (I) is one where the quantity of at least one component of the base alloy is increased or decreased, at least one ductility component is incorporated into the base alloy, or both, with these three options being referred to as “alloy modification” herein.

In embodiments where a ductility component is incorporated into the base alloy to improve ductility, suitable ductility components include any element other than noble gases as well as mixtures of elements other than noble gases, such as metals (alkali metals, alkaline earth metals, transition metals), metalloids, interstitial elements, or mixtures thereof. Examples include one or more of cobalt, lithium, sodium, potassium, rubidium, cesium, francium, beryllium, magnesium, calcium, strontium, barium, radium, scandium, yttrium, titanium, zirconium, hafnium, rutherfordium, vanadium, niobium, tantalum, dubnium, chromium, molybdenum, tungsten, seaborgium, manganese, technetium, rhenium, bohrium, ruthenium, osmium, hassium, rhodium, iridium, meitnerium, nickel, palladium, platinum, darmstadtium, copper, silver, gold, roentgenium, zinc, cadmium, mercury, copernicium, aluminum, gallium, indium, thallium, nihonium, tin, lead, flerovium, bismuth, moscovium, polonium, livermorium, tennessine, boron, silicon, germanium, arsenic, antimony, tellurium, astatine, carbon, oxygen, sulfur, phosphorus, or mixtures thereof.

In some embodiments, the ductility component will be the same as one already present in the base alloy (e.g., adding vanadium to a base alloy comprising iron, cobalt, vanadium, and possibly minor impurities). Thus, adding the ductility component to the base alloy will be equivalent to adjusting the quantity of a component (e.g., first metal, chemical element different from first metal, and/or the additional component) already present in that base alloy. In other embodiments, the ductility component will be entirely new to the base alloy (e.g., adding vanadium to a base alloy that includes only iron, cobalt, and possibly minor impurities).

Regardless, the amount of ductility component can be selected and adjusted depending on the base alloy, the base alloy's properties, and/or the properties desired in the final alloy. In one or more embodiments, the ductility component (e.g., additive metal) can be added to the base alloy at levels up to about 20 at. %, preferably up to about 15 at. %, more preferably up to about 10 at. %, even more preferably up to about 5 at. %, and most preferably up to about 3 at. % so as to identify a modified base alloy (I) for modeling. Additionally or alternatively, the quantity of an existing component of the base alloy can be increased or decreased by up to about 20 at. %, preferably up to about 15 at. %, more preferably up to about 10 at. %, even more preferably up to about 5 at. %, and most preferably up to about 3 at. % so as to identify a modified base alloy (I) for modeling. If desired, the ductility component and/or existing component quantities can be adjusted incrementally (e.g., by about 0.3 at. % to about 5 at. %, preferably about 0.5 at. % to about 3 at. %, and more preferably about 0.5 at. % to about 2 at. %), as many times as necessary until the desired ductility would achieved. In any instance, the at. % is based on the total at. % of the base alloy taken as 100%.

It will be appreciated that the above-described alloy modification process can also be carried out on modified base alloy (I) to identify a modified base alloy (II) whose strain accommodation energy and cleavage energy are compared to determine whether the modified base alloy (II) would achieve the predetermined difference in those energies. If the predetermined difference in those energies would not be achieved in modified base alloy (II), the alloy modification process can be repeated on modified base alloy (II), thus identifying a modified base alloy (III) whose strain accommodation energy and cleavage energy are compared to determine whether the predetermined difference in energies is achieved by modified base alloy (III), and so on. Thus, this process can be repeated as many times as necessary or desired until a modified alloy that is sufficiently ductile (referred to as “final alloy”) has been identified.

Preferably, the predetermined difference in energies comprises a cleavage energy that is greater than the strain free energy (i.e., the energy of the material with no added strain), more preferably about 2% greater, even more preferably about 5% greater, and most preferably about 10% greater.

In some embodiments, the predetermined difference in energies comprises a cleavage energy that is greater than the strain accommodation energy, preferably about 2% greater, more preferably about 5% greater, and even more preferably about 10% greater.

In one or more embodiments, the strain accommodation energy at the time of cleavage is at least about 2 J/m², preferably at least about 3 J/m², and even more preferably about 3 J/m² to about 20 J/m².

b. Final Alloy Identified and Formed

Once a final alloy has been identified (be it the proposed base alloy or a modified version of the base alloy identified as described above), that final alloy can be formed according to the identified compositional makeup of that final alloy. Conventional alloying processes can be used to form the final alloy, including powder metallurgy processes, melting processes such as casting, or melt atomization such as spray, impulse, or gas atomization.

Powder metallurgy generally comprises forming the various alloy components (e.g., first metal, chemical element different from the first metal, any additional component, and/or any ductility component) into a powder. The powders can then be blended (e.g., in a screw mixer, rotary drum, etc.) with other powders and/or any binders, lubricants, and/or other additives needed to achieve the desired properties. The powder blends are then compacted under high pressures (e.g., about 80 MPa to about 1,600 MPa) for time periods sufficient to form a substantially void-free, dense compact (e.g., about 10 seconds to about 5 minutes). The compact is then sintered at sufficient temperatures and times to cause the loose particles to bond and form a solid piece. The sintering temperature is typically about ⅔ to about ¾ of the melting temperature of the composition being sintered. Sintering times are typically about 60 minutes to about 150 minutes. Powder metallurgy processes include powder forging, hot isostatic pressing, metal injection molding, electric current assisted sintering, and/or additive manufacturing.

Casting processes broadly involve mixing metals in a molten state and then pouring that mixture into a mold (e.g., metal mold, sand mold) and allowing that mixture to solidify. One skilled in the art can select the temperatures for forming the components into a molten mixture and the order of melting the various alloy components based on their respective melting points and concentrations.

Melt atomization methods involve forming a stream of melted alloy components and causing those components to form a dispersion of fine particles, typically using a spray of gas or liquid. Those particles are solidified into metal droplets and/or powders, typically by allowing them to fall and cool as they make contact with a substrate such as a metal plate, followed by collection of the formed alloy powder.

Regardless of the alloying process, in some embodiments, the final alloy will comprise the ductility component (e.g., additive metal) at an atomic level and/or weight level that is lower than that of the first metal. Alternatively or additionally, the final alloy will comprise the ductility component (e.g., additive metal) at an atomic level and/or weight level that is lower than that of the chemical element different from the first metal.

In instances where the final alloy contains four or more total components (i.e., first metal, chemical element different from the first metal, ductility component, and additional component), the first metal and the chemical element different from the first metal will generally be the two that are present in the final alloy at the two highest atomic percentages. It is preferred that the combined at. % of the first metal and of the chemical element different from the first metal comprise at least about 80 at. %, preferably at least about 90 at. %, more preferably about least about 95 at. %, and most preferably about 95 at. % to about 98 at. %, based on the total at. % of the final alloy taken as 100%.

Thus, in embodiments where the final alloy includes both a ductility component(s) and an additional component(s), it is preferred that the total quantity (atomic % and/or weight %) of the ductility component(s) and the additional component(s) is less than the total quantity (atomic % and/or weight %) of the first metal and/or the total quantity (atomic % and/or weight %) of the chemical element different from the first metal.

The respective quantities in the final alloy of the first metal and the chemical element different from the first metal are not generally limited and will be determined, at least in part, by the base alloy and the alloy modification process. However, in some embodiments, it is preferred that the chemical element different from the first metal is present in the alloy in a quantity that is within about 20 at. %, preferably within about 15 at. %, more preferably within about 10 at. %, even more preferably within about 5 at. %, and most preferably within about 2 at. % of the quantity of the first metal. In one or more embodiments, the atomic ratio of the first metal to the chemical element different from the first element in the base alloy is about 0.8 to about 1.2, preferably about 0.9 to about 1.1, and even more preferably about 1:1.

Additionally or alternatively, in some embodiments, the ductility component(s) is present in the final alloy at a level up to about 20 at. %, preferably up to about 15 at. %, more preferably up to about 10 at. %, even more preferably up to about 5 at. %, and most preferably up to about 3 at. %., with the at. % being based on the total at. % of the base alloy taken as 100%.

One preferred final alloy comprises iron, cobalt, and vanadium.

Another preferred final alloy comprises about 48 at. % to about 51 at. % iron, about 48 at. % to about 51 at. % cobalt, and about 1.05 at. % to about 2.25 at. % vanadium.

A further preferred final alloy comprises about 48.40 at. % to about 50.10 at. % iron, about 48.10 at. % to about 49.55 at. % cobalt, and about 1.70 at. % to about 2.15 at. % vanadium.

In each of the base alloy, modified alloy, and/or final alloy, minor impurities may be present, typically at levels of about 0.5 at. % or lower, preferably about 0.4 at. % or lower, more preferably about 0.2 at. % or lower, even more preferably about 0.1 at. % or lower, and most preferably about 0 at. %.

It will be appreciated that the above process can be used to modify an intermetallic or other base alloy that has a property(ies) desirable for a particular end use but which is not sufficiently ductile for that end use. One skilled in the art can use the above process to identify potential modifications to that base alloy that would yield a final alloy that is sufficiently ductile while largely or completely retaining the other property(ies) that made the starting base alloy desirable in the first place.

Additional advantages of the various embodiments will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments but is not necessarily included. Thus, the present disclosure encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the disclosure. It is to be understood, however, that these examples are provided by way of illustration, and nothing therein should be taken as a limitation upon the overall scope.

### Example 1

### Ductility Modeling

### 1. Methods

Density functional theory (DFT) calculations were used to develop fundamental methods to understand the ductility of intermetallics. Vienna Ab initio Simulation Package (“VASP”), version 5.4, was used within the MedeA software. (Materials Design, I. MedeA® Software. (1998).) All calculations in this work leverage the projector augmented-wave (“PAW”) method. (Blochl, Projector augmented-wave method, Phys. Rev. B 50, (1994); Kresse et al., From Ultrasoft Pseudopotentials to the Projector Augmented Wave Method, Phys. Rev. B 59, (1999).) Exchange-correlation effects were evaluated using the generalized gradient approximation (“GGA”) (Perdew et al., Generalized Gradient Approximation Made Simple. Phys. Rev. Lett. 77, 3865 (1996); Perdew et al., Atoms, Molecules, Solids, and Surfaces: Applications of the Generalized Gradient Approximation for Exchange and Correlation. Phys. Rev. B 46, 6671 (1992); and Perdew et al., Erratum: Atoms, Molecules, Solids, and Surfaces: Applications of the Generalized Gradient Approximation for Exchange and Correlation. Phys. Rev. B 48, 4978 (1993)) with the Perdew, Burke, and Ernzerhof (“PBE”) functional with spin-orbit coupling and produced only a small error, 0.322%, in calculating the lattice parameter of B2 Fe—Co. (Perdew et al., Restoring the Density-Gradient Expansion for Exchange in Solids and Surfaces. Phys. Rev. Lett. 100, 136406 (2008).)

While it is generally true that k-points must be converged for each different atomistic geometry, and the energy cutoff must be converged whenever the elements present in a system change, there can be some interaction between these two parameters and each new system should have both values converged simultaneously. In order to ensure that different material chemistries are not being evaluated with different basis sets, a single set of k-point spacing and energy cut-off values was selected for each structure (Table 1). These values were chosen using a convergence criteria of 0.3 eV/atom and were checked with and without vanadium present.

TABLE 1
Converged Simulation Parameters for Each Atomistic Structure.

| | k-point Spacing | Energy Cut-off |
|---|---|---|
| Models | (1/Ang) | (eV) |
| {110} APB and Cleavage | 0.1458 | 750 |
| {112} APB | 0.1458 | 750 |
| {310} GB and Cleavage | 0.234 | 750 |

Every structure was geometrically relaxed to 0.02 eV/Å and then the energy evaluated to an accuracy of 1E-5 eV. All anti-phase boundaries, grain boundaries, and surfaces were relaxed with fixed lattice dimensions to approximate the constraints of the surrounding system. This generates the assumption that the individual defects modeled here are far enough apart to avoid the creation of significant interactions. The fixed lattice parameters are individually based on the relaxed lattice with matching chemical defects.

The various chemistries and defects reported are all generated within the same three basic structures: a 64-atom cell with the <110> direction along the z-axis, a 96-atom cell with the <112> direction along the z-axis, and a 120-atom cell with a {310} grain boundary with the <310> direction along the z-axis. APBs are created by altering the ordering within the 64-atom cells, and 96-atom cells and surfaces are created by removing the atoms on the top half of the cell and leaving a vacuum. Alterations in stoichiometry can be achieved in any of these cells by substitution, the introduction of vacancies, or a combination of the two at specific sites within the structure. Alterations in stoichiometry and the stabilities of individual defects are explored in this work.

Dislocation energy calculations leveraged a number of values to enable calculations of dislocation density (Table 2). The dislocation length was assumed to be 25 nm, approximately the crystallite size in the material. Any error in crystallite size could lead to differences in dislocation energies, however, the dislocation length has a linear relationship with dislocation energy and, for the purposes of this work, the relative differences between two materials would simply be proportional to the dislocation length. Dislocation travel “x” was held constant at 25 μm to enable comparison.

TABLE 2
Selected Variable Values Used in Calculations
of Dislocation Density and Energy.

| Variable | Selected Value | |
|---|---|---|
| Dislocation length, L | 25 | μm |
| Burgers vector, b | 2.4687 | Å |
| Shear modulus, G | 80 | GPa |
| Dislocation core radius, rₒ | 3.703 | Å |
| Dislocation Travel, x | 25 | μm |

### 2. Model Development

The basis of this model is the theory that the ductility of a material is governed by a competition between (1) the accommodation of strain and (2) the initiation of cleavage, which leads to the development of a ductility and cleavage model that is sensitive to changes in chemistry and can predict the relative strains at which quasi-static room temperature cleavage will occur. To incorporate the chemistry sensitivity, DFT models are used to determine relative APB and surface energies and atomic interactions in various structures. APB energies are then used to calculate the energy necessary to cross-slip, which is used as a modifier for the total energy needed to accommodate strain, while surface energies are used to determine the energy necessary to initiate cleavage. These energy values, informed by the DFT models described above, are then compared to determine the strain at which the strain accommodation energy and cleavage energy are equivalent, which represents the critical point at which cleavage begins.

This DFT approach also allows for comparisons of the stability of various defects but will not provide overall phase stability data. For example, comparison of defect energies can indicate the relative stabilities of substitutional defects in various lattice positions, such as on grain boundaries or APBs, but does not indicate if an additional phase has formed with the altered chemistry. For example, cobalt-rich Fe—Co alloys with vanadium can form additional intermetallic phases such as the (Fe,Co)₃V phase. This model does not account for additional phase formation, but one skilled in the art would be aware of that possibility and could account for it.

a. Strain

The accommodation of plastic strain in inorganic materials at quasi-static strain rates and room temperature is largely comprised of the creation and motion of dislocations within the structure. The model for the energy of a screw dislocation used was Equation (1).

$$Γ = \frac{{Gb}^{2}}{4 π} ln ( \frac{R}{{r}_{0}} ) \qquad ( 1 )$$

where G is the shear modulus of the material, b is the burger's vector, rₒ is the dislocation core radius, and R is the cut-off of the elastic field around the dislocation. By assuming an average dislocation length, relating the cut-off of the elastic field to dislocation density with R=1/(2\sqrt{ρ}), and multiplying the individual dislocation energy by the density of dislocations, total dislocation energy was calculated using Equation 2.

$${E}_{D} = ρ L \frac{{Gb}^{2}}{4 π} ln ( \frac{1 / ( 2 \sqrt{ρ} )}{{r}_{0}} ) \qquad ( 2 )$$

where ρ is the dislocation density, and L is the average dislocation length. With this equation, the incremental change in total dislocation energy can be determined as dislocations are added to the system. This approach provides a simple way to evaluate relative tolerance of a given material to more dislocations.

DFT was used to calculate the APB energies for cross-slip from a {110} plane to a {112} plane, which is the lowest energy path for cross-slip of Fe—Co alloys. To incorporate the impact of cross-slip on dislocation energies, an equation developed by Paidar to calculate the shear stress necessary to cross-slip as a function of APB energy was used. (Paidar, Cross-slip of Superpartial Dislocations in Iron Aluminides, Philos. Mag. A Phys. Condens. Matter, Struct. Defects Mech. Prop. 81, 1065-1077 (2001).) By solving for the integral of the shear stress equation with respect to cross-slip distance, the energy necessary to cross-slip (E_{CS}) was determined. Cross-slip energy is then used to modify the overall dislocation energy by directly adding the two area normalized energies. This results in an overall reduction in energy per dislocation if cross-slip energy is negative.

b. Cleavage

For the purposes of this model, only intergranular and transgranular cleavage were considered; microvoid coalescence was not considered due to the low likelihood that the ductility of ordered Fe—Co can be increased enough to make it relevant. Intergranular cleavage, the failure of a material preferentially along grain boundaries, and transgranular cleavage, the failure of a material through a grain, are both brittle failure modes occurring at low strains and can be approximated through the application of Griffith's law (Equations 3 & 4).

E_{TC}=2γₛ−E_{APB} (3)

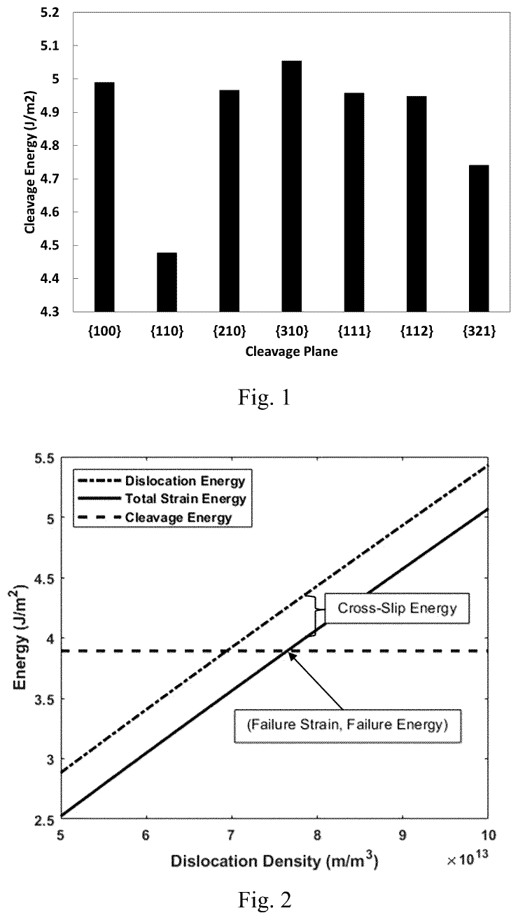

E_{IC}=2γₛ−E_{GB} (4)

Where E_{TC} and E_{IC} are the transgranular and intergranular cleavage energies, respectively, γₛ is the energy of the surface created during cleavage, E_{APB} is the APB energy, and E_{GB} is the grain boundary energy. These equations allow for the calculation of necessary cleavage energies by performing DFT calculations on cohesive grains, grain boundaries, APBs, and surfaces. For transgranular cleavage, determining the most likely crystallographic plane for cleavage, i.e., the plane with the lowest energy cleavage surface, is needed to enable correct comparison of intergranular and transgranular cleavage; this plane was determined to be the {110} plane through a survey of various crystallographic planes in equiatomic Fe—Co (FIG. 1). Unlike transgranular cleavage, the number of possible grain boundaries for intergranular cleavage is far too numerous to survey for an ideal candidate so a mirror tilt grain boundary was used for simplicity; since the {310} boundary has approximately 37 degrees of misorientation, it is considered a high-angle grain boundary and is assumed to reasonably represent the case of intergranular cleavage.

The transition point between intergranular and transgranular cleavage can be determined using the previously calculated cleavage energies (Equations 3 and 4 above) and following Equation 5.

M_{F}=E_{TC}−E_{IC}E_{CS} (5)

where M_{F} is the fracture mode criterion, E_{TC} and E_{IC} are the transgranular and intergranular cleavage energies, respectively, and E_{CS} is the cross-slip energy. This criterion calculation creates a balance between the energy necessary to cross-slip, or activate additional slip planes, and the energetic difference between the two cleavage modes. If the cross-slip energy is more negative than the difference between the two cleavage modes, i.e., M_{F} is less than zero, then the cleavage mode changes from intergranular to transgranular.
c. Strain to Failure

All of the above techniques are then compiled using energetic competition between the accumulation of dislocations and the predicted cleavage mode. Referring to FIG. 2, the dislocation energy is modified by the negative cross-slip energy to produce a lower total strain energy (used interchangeably with strain accommodation energy). The cleavage energy plotted is whichever cleavage energy applies based on the result of Equation 5 above. The point where total strain energy and cleavage energy intersect represents the strain to failure for the system. The dislocation density, where dislocation energy and the energy of the predicted cleavage mode are equivalent, is the point at which failure begins. When comparing two chemistries, the change in the dislocation density at which failure begins can be related to a change in strain to failure through Equation 6.

Δε=Δρbx (6)

where Δε is the change in strain and x is the distance the average dislocation has moved through the crystal. This approach to relating strain and dislocation density enables a more useful comparison of different materials than dislocation density alone by enabling a relation to either the dislocation density in a strain-relieved polycrystal or the strain to failure of an alloy of interest. In this case, the cleavage strain for equiatomic Fe—Co is assumed to be zero and increased strain to failure of other alloys will be compared to the range of reported values in literature. This model results in both a prediction of strain to failure and the preferred cleavage mode, thus providing a comparison between the strain accommodation energy and the cleavage energy so that one can determine whether the desired difference in the two would be achieved with the modeled alloy so that the modeled alloy would be sufficiently ductile.

### 3. Results

A number of evaluations were performed to bound the problem set and determine general defect stabilities in Fe—Co alloys. First, to ensure that lower rigor atomic potentials provide valid results, a number of simulations were performed using different atomic potentials and compared to available literature. Second, a fundamental investigation of the basic point defect stabilities relative to each other and to other defects in the structure was performed to understand what types of defects should be expected. Finally, several cross-slip and cleavage energies were calculated and incorporated into the overall model proposed above to determine relative strain to failure as a function of chemistry. All of these results are discussed in the context of available knowledge about the Fe—Co alloys and their respective ductilities.

a. Effect of Potentials With Higher Valence

VASP offers a series of potentials with increasing valence, i.e., incorporation of p and s orbitals. Outer orbitals such as the d orbital in transition metals tend to dominate the interactions between atoms, but can still be influenced by inner orbitals so the inclusion of s and p orbitals may lead to more accurate solutions for some models but increases the computational cost (Table 3). Relevant to this work, VASP recommends using the lowest valence potentials for iron and cobalt and the highest valence potentials for vanadium. When using these recommendations, the predicted energy of a {110} APB in stoichiometric Fe—Co is 0.125 J/m². This value fits within the range of values reported in literature but is lower than recent values reported using DFT with ultrasoft potentials. VASP recommends the use of its projector augmented-wave (“PAW”) potentials as opposed to ultrasoft potentials due to smaller radial cutoffs and reconstruction of the exact valence wave function.

TABLE 3
Default cutoff energies and valence for iron, cobalt,
and vanadium PAW potentials, where PAW_pv incorporates p
orbitals and PAW_sv incorporates s and p orbitals.

| | | Default cutoff Energy | |
|---|---|---|---|
| Element | Potential | (eV) | Valence |
| Fe | PAW | 268 | 8 |
| | PAW_pv | 293 | 14 |
| | PAW_sv | 391 | 16 |
| Co | PAW | 268 | 9 |
| | PAW_pv | 271 | 15 |
| | PAW_sv | 390 | 17 |
| V | PAW | 193 | 5 |
| | PAW_pv | 264 | 11 |
| | PAW_sv | 264 | 13 |

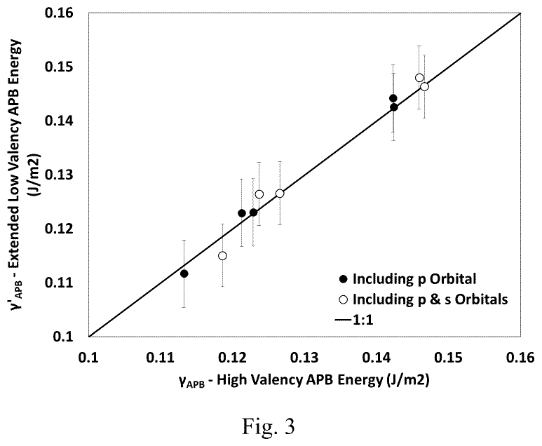

To investigate these differences, the {110} APB energies for the systems of interest were calculated using each of the available potentials for iron and cobalt. All calculations that included vanadium used the highest valence potential for vanadium atoms. A clear trend appeared for the change in system energies, with the PAW_pv potential increasing energy per atom by 0.299±0.003 eV/atom in systems with no APB and by 0.279±0.004 eV/atom in systems with an APB. Similarly, system energies with the PAW_sv potential increased by 1.77±0.003 eV/atom in systems with no APB and 1.75±0.004 eV/atom in systems with an APB. Because of the highly consistent trend with increasing valence, these energy increases were used to predict the APB energy with higher valence potentials (FIG. 3). Results show that a simple extrapolation of the PAW results to higher absolute APB energy values closely predicts the values produced by the higher valence calculations with PAW_pv and PAW_sv potentials. This is a strong indication that the true APB energy is likely higher than the values calculated using the lower valence PAW calculations. However, the trends are the same both with and without higher valence potentials; as such, for the remainder of the analysis in this work, only the low valence calculation will be used.

b. Point Defect Stability

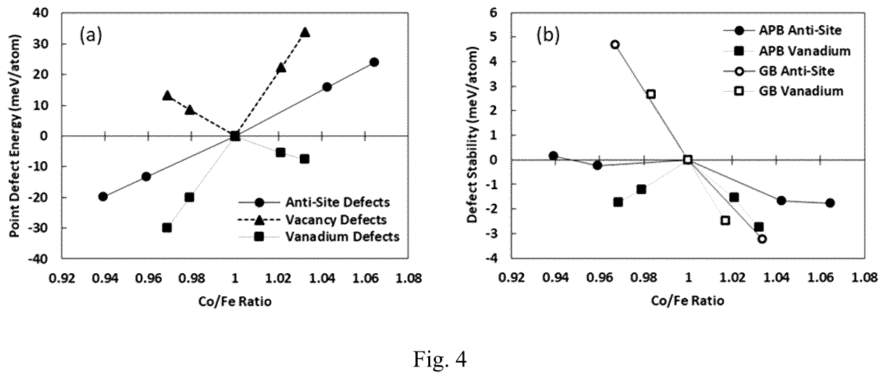

In order to further validate the developed modeling approach, as well as to address unanswered questions about point defect stabilities both with and without vanadium, a series of models of 64 and 96 atoms were completed to determine the energy of vacancy, anti-site, and vanadium substitution defects. Results for the Fe—Co system indicate that anti-site defects are stable on both sides of stoichiometry, and that vanadium substitutions are stable across all chemistries evaluated but prefer to be located on iron sites (FIG. 4(a)). Since vacancy defects are not thermodynamically stable and the highly ordered material that is desired for magnetic performance requires slow cooling during heat treatment (which promotes minimal vacancy formation), vacancies were ignored for the remaining analysis.

When evaluating the stability of a given chemical variation at APBs or grain boundaries, the point defect energy was calculated with the boundary present and compared to the energy without the boundary present (FIG. 4(a)) to determine the energy the defect at the boundary. FIG. 4(b) shows the relative stability of point defects at both APBs and grain boundaries; anti-site defects are stable at APBs for all chemistries, especially cobalt-rich materials, though vanadium substitutions are more stable in all cases. These results indicate that it is reasonable for anti-site defects and vanadium substitutions to be present on both {110} and {112} APBs in the chemistry ranges evaluated.

Point defect stability at grain boundaries behaves differently than at APBs and has a stronger dependency on chemistry. Both anti-site defects and vanadium substitutions on grain boundaries are unstable in cobalt-lean materials and stable in cobalt-rich materials. This indicates that anti-site defects and vanadium substitutions would not be expected to appear on grain boundaries in cobalt-lean materials but would appear on grain boundaries in cobalt-rich materials, driving a change in the grain boundary energy. It should be noted that the relative energy values are a fairly low driving force for chemical segregation to APB's and grain boundaries, but are in an appropriate range of values, thus indicating that the calculations are likely accurate, and the effects observed are minimal.

The likely defect population of commercial materials would be mostly vanadium substitutions on cobalt sites with some iron substitutions on cobalt sites and slight segregation of vanadium defects to APBs. These results emphasize the criticality of understanding the effects of slight changes in stoichiometry, especially when designing any dopant chemistries for intermetallics.

### 4. Discussion

a. Anti-Phase Boundaries and Cross-Slip

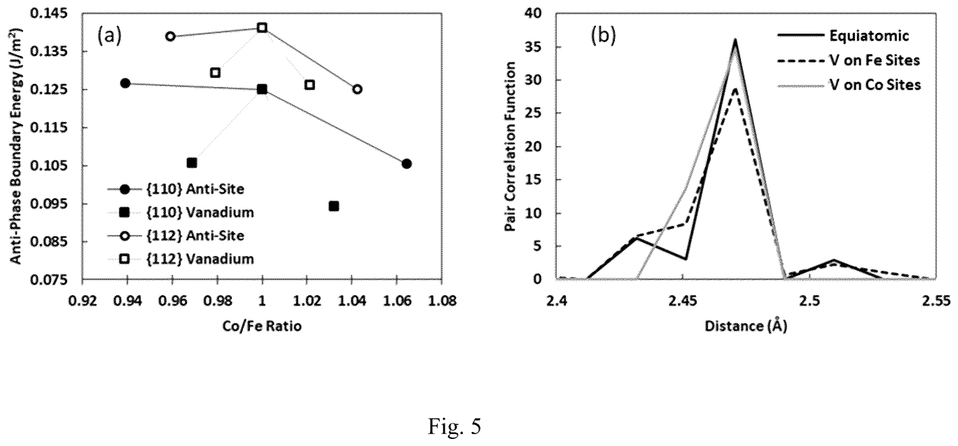

Since all of the point defect variations for this work are stable at APBs, the potential impact of such defects on the APB energies and the energy necessary to cross-slip may play a role in the overall ductility of the material. FIG. 5(a) shows the APB energies on both {110} and {112} planes. Vanadium lowers the APB energy in all cases, and APB energies overall appear to be lowest in cobalt-rich material. The observed changes in energy appear to be the result of changes in boundary ordering. FIG. 5(b) shows the relative spacing of atoms around the {110} APBs with and without the addition of vanadium there is a broadening of the relative spacings when vanadium is added, especially in cobalt-rich materials, which indicate disordering. Additionally, the {112} APB energies are higher than the {110} APB energies in every case, which confirms that dislocations would form first on the {110} planes and would move from the {110} planes to the {112} planes through cross-slip.

To begin translating atomistic model results to predict the properties of real materials, the DFT model results were generalized to any stoichiometry. Due to the high computational cost of DFT models, only two data points were determined for each case, and thus only a series of simple linear models could be determined. Those linear models are compiled additively as shown in Equation 7.

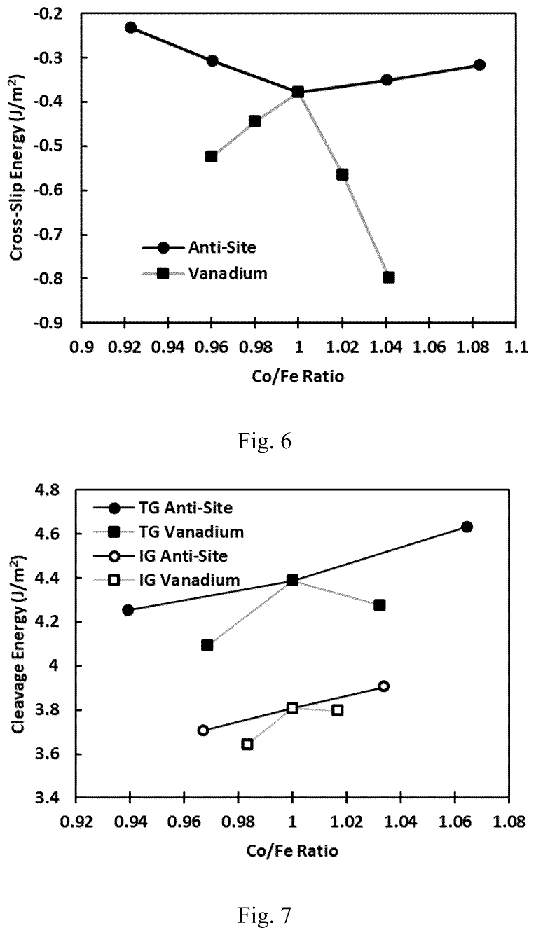

E′_{APB}=E_{APB}+α₁[V_{Co}]+α₂[V_{Fe}]+α₃[Fe_{Co}]+α₄[Co_{Fe}] (7)

where E′_{APB} is the modified APB energy, and E_{APB} is the equiatomic APB energy. The linear models are α₁, α₂, α₃, and α₄, and they are multiplied by the concentration of the subject element that is located on the equiatomic site denoted by the subscript. The relative cross-slip energies calculated using the APB energies from Equation 7 are shown with respect to their stoichiometry in FIG. 6. The calculated cross-slip energy depends on the energy difference between the equiatomic and modified APB energies for a given chemistry. For example, even though the APB energy decreased from equiatomic Fe—Co on both planes with the introduction of cobalt-rich anti-site defects, the energy necessary for cross-slip increased. This is due to the larger decrease on the {112} plane relative to the decrease on the {110} plane with the same chemical variation. Vanadium additions exhibit the opposite behavior, with the APB energy decreasing more significantly on the {110} plane, thus driving an overall decrease in the energy necessary for cross-slip.

Assuming no change in cleavage energy with a given type of defect in the material relative to the intergranular fracture energy for equiatomic Fe—Co, the change in strain to failure for 2 at. % vanadium substituted in cobalt-rich material and in cobalt-lean material, calculated using the overall model, is 2.04% and 0.69%, respectively. While these values do not indicate enough improvement in strain to failure to completely explain the observed ductility in these alloys, they do indicate that changes in APB energy, and thus cross-slip, are likely a key contributing factor. When considering commercial cobalt-lean alloys, some improvement in the cross-slip energy can be expected due to vanadium substitutions, though this would likely be partially counteracted by the smaller increase in cross-slip energy driven by the anti-site defects that would be present. It appears that vanadium additions to cobalt-rich material, not the typical cobalt-lean material, would provide a more significant impact to cross-slip and ductility.

b. Intergranular and Transgranular Cleavage

The energy necessary to cleave {310} grain boundaries and {110} planes can be seen in FIG. 7. There was a trend of higher cleavage energies in cobalt-rich materials and lower cleavage energies with the presence of vanadium. The overall changes in all cleavage energies were all under 5%, which is much smaller than the calculated changes in cross-slip energies. The reduced intergranular cleavage energies in cobalt-lean materials can be, and was, ignored due to the previously discussed lack of point defect stability at grain boundaries with this stoichiometry.

A similar linear model to that was used for APB energies above can be applied for each case of cleavage energy and is shown in Equations 8 and 9.

E′_{TC}=E_{TC}+φ₁[V_{Co}]+φ₂[V_{Fe}]+φ₃[Fe_{Co}]+φ₄[Co_{Fe}] (8)

E′_{IC}=E_{IC}+φ₁[V_{Co}]+φ₂[V_{Fe}]+φ₃[Fe_{Co}]+φ₄[Co_{Fe}] (9)

Where E′_{TC} and E′_{IC} are the modified cleavage energies, and E_{TC} and E_{IC} are the equiatomic cleavage energies. The relative linear models are φ₁, φ₂, φ₃, φ₄, ω₁, ω₂, ω₃, and ω₄, and they are multiplied by the concentration of the subject element that is located on the equiatomic site denoted by the subscript.
c. Strain to Failure

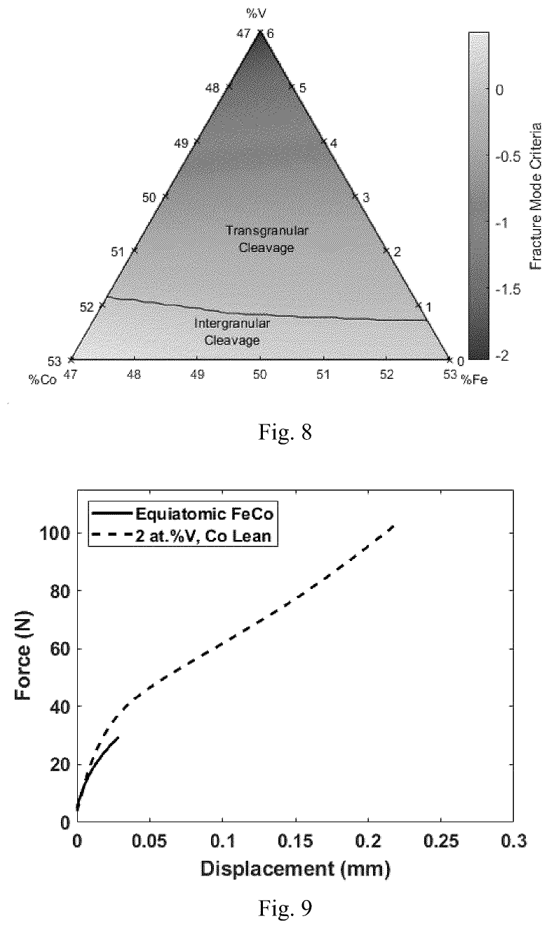

When leveraging the linear models constructed from the simulation results (Equations 7, 8, and 9), the cleavage mode can be determined as a function of stoichiometry and vanadium additions with Equation 5 (FIG. 8). Even adding 2 at. % vanadium to cobalt-lean and cobalt-rich materials using the overall model increased the cleavage strain by 1.70% and 4.18%, respectively. In both cases, transgranular cleavage is predicted. These values are in the range of values reported in literature, though the significant difference between the two values has yet to be rigorously explored experimentally.

### 5. Conclusions

The above analysis provides significant insight into the deformation mechanisms in ordered materials such as Fe—Co alloys, and provides a basis for exploring new chemistries and materials. APB energies and cleavage energies improved in cobalt-rich materials, both with and without vanadium present. Vanadium reduced the energy required to cross-slip super-dislocations and reduced cleavage energies. The developed model predicted both the historically observed increases in ductilities, and the transition of cleavage mode from intergranular to transgranular cleavage with the vanadium additions.

### Example 2

### Disk Bend Testing

In this Example, the modeling developed in Example 1 was tested by fabricating a series of Fe—Co alloys, both with and without vanadium, as well as with different stoichiometries. Those alloys were then subjected to disk bend testing and microstructural analysis.

### 1. Methods

High purity, −325 mesh iron, cobalt, and vanadium powders were weighed to an accuracy of 0.001 grams and mixed to form the various alloys listed in Table 4. A simple cylindrical die set was then volume filled with the powder mix and pressed at room temperature to approximately 35 MPa to form briquettes. Next, several briquettes at a time were arc melted using a tungsten arc welder inside a bell jar on a water chilled copper plate. The bell jar was evacuated and backfilled with argon three times to ensure minimal oxygen exposure, and the sample was melted, inverted, and re-melted three times. Chemistry testing was performed on approximately one-gram pieces of the arc melted samples. Bulk chemistry was performed using acid digestion followed by inductively coupled plasma atomic emission spectrometry (ICP-AES) and inert gas fusion per American Society for Testing and Materials (ASTM) E1019-18.

Table 4 shows the chemistry results for the arc melted samples. In most cases, the measured chemistry was close to the targeted values. However, the 1 at. % vanadium, cobalt-lean alloy had higher-than-targeted vanadium content, and all cobalt-rich alloys had lower than targeted cobalt content. While these differences between target stoichiometries and actual stoichiometries could drive some minor imbalance to the analysis of the results, they still provide a good test of the linear model. Oxygen content was measured and did not exceed 0.5 at. %. Since all materials were heat treated in a reducing environment after chemistry tests were performed and prior to disk bend sample preparation, it is possible that oxygen values were even lower in the tested disk bend samples. Vanadium-free samples were observed to have a significantly larger grain size than the vanadium-containing alloys, likely due to some form of grain pinning during solidification.

TABLE 4
Chemistry results for arc melted samples compared
to the target chemistries (shown in parenthesis).

| | Co | Fe | V | O | V Error | Target | | Error |
|---|---|---|---|---|---|---|---|---|
| Alloy | (at. %) | (at. %) | (at. %) | (at. %) | (%) | Co/Fe | Co/Fe | (%) |
| Equiatomic | 49.74 (50) | 49.78 (50) | — | 0.48 | — | 1.00 | 1.00 | 0.08 |
| 2 at. % Co-rich | 50.55 (52) | 49.02 (48) | — | 0.43 | — | 1.08 | 1.03 | 4.81 |
| 1 at. % Co-rich | 50.64 (51) | 49.19 (49) | — | 0.18 | — | 1.04 | 1.03 | 1.09 |
| 1 at. % Co-lean | 49.16 (49) | 50.63 (51) | — | 0.21 | — | 0.96 | 0.97 | 1.07 |
| 2 at. % Co-lean | 48.15 (48) | 51.75 (52) | — | 0.10 | — | 0.92 | 0.93 | 0.80 |
| 2 at. % V, Co-rich | 49.43 (50) | 48.33 (48) | 2.09 (2) | 0.15 | 4.43 | 1.04 | 1.02 | 1.80 |
| 1 at. % V, Co-rich | 49.22 (50) | 49.58 (49) | 1.08 (1) | 0.11 | 7.93 | 1.02 | 0.99 | 2.72 |
| 1 at. % V, Co-lean | 48.06 (49) | 49.92 (50) | 1.77 (1) | 0.25 | 77.18 | 0.98 | 0.96 | 1.76 |
| 2 at. % V, Co-lean | 48.25 (48) | 49.4 (50) | 2.22 (1) | 0.13 | 11.16 | 0.96 | 0.98 | 1.74 |

All arc melted samples were put through an ordering heat treatment at 860° C. for 4 hours following final sample preparation and validation of acceptable stoichiometry. All heat treatments were completed in a hydrogen environment to minimize the risk of oxidation. Disk bend samples were prepared using an electrical discharge machining to section a thin region from the melted samples, and the sectioned regions or “wafers” were then polished to a thickness of 0.25 mm. Disks with a diameter of 3.0 mm were punched from the polished wafer using a transmission electron microscopy (TEM) punch. Samples were placed into a set of disk bend fixtures made internally as described by Manahan, M. P. et al., The Development of a Miniaturized Disk Bend Test for the Determination of Postirradiation Mechanical Properties, Journal of Nuclear Materials, 104, 1545-1550 (1981). The samples were bent at a displacement rate of 0.0003 in/sec (0.00762 mm/sec) with a load cell and deflectometer. Scanning electron microscopy (SEM) was used on a subset of the broken disk bend samples to determine the cleavage mode which were then laser sectioned, mounted, and polished to their midpoint and evaluated with electron backscatter detection (EBSD).

All disk bend test samples performed in two characteristic ways as shown in FIG. 9, with vanadium-containing alloys exhibiting significantly higher failure displacements than the vanadium-free alloys. The initial high loading rate region for all samples appears to represent the elastic bending region commonly reported for disk bend testing. Samples without vanadium did not extend significantly beyond this region, but samples with vanadium transitioned into the plastic bending region, and possibly the membrane stretching region, before failure. As would be expected for a brittle material failure, which exhibits no necking, there was no significant reduction in loading rate prior to failure. Overall, disk bend tests proved highly effective at distinguishing between different levels of ductility in the samples evaluated.

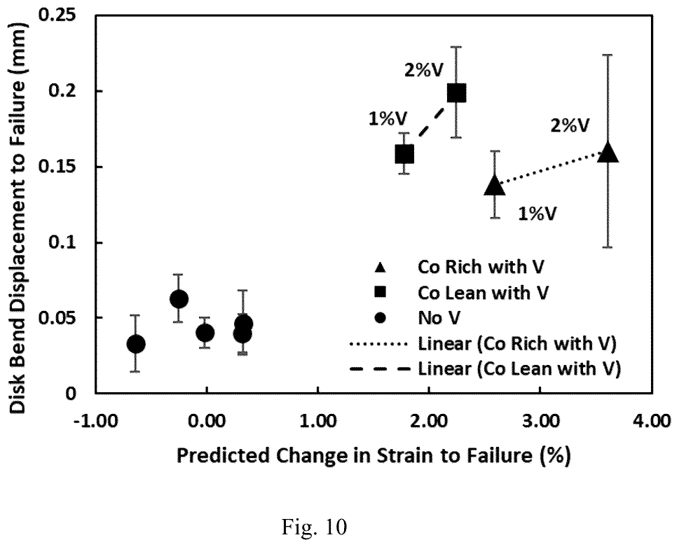

To effectively use the experimental disk bend results to validate the model, failure strain predictions were made for each of the experimental alloy's measured chemistry, as reported above in Table 4. FIG. 10 shows the relationship between the model predictions and the disk bend results for each alloy, with lines representing a linear regression for an alloy group, and error bars in this (and all graphs herein) representing the 95% confidence interval for the standard error of the mean. Overall, as predicted, the disk bend results showed that additions of vanadium drove a significant increase in the displacement to failure. Alloys without any vanadium showed no statistical difference in displacement to failure as a function of stoichiometry despite the model predicting some minor changes to failure strain. Two distinct relationships between the model predictions and the experimental results appeared for alloys with vanadium with cobalt-lean alloys exhibiting lower predicted failure strain and higher measured performance due to the formation of a new phase, as described previously in Example 1, Part 2. If adjustments are made for that new phase, the relationship between predictions and the disk bend results fall on a single line.

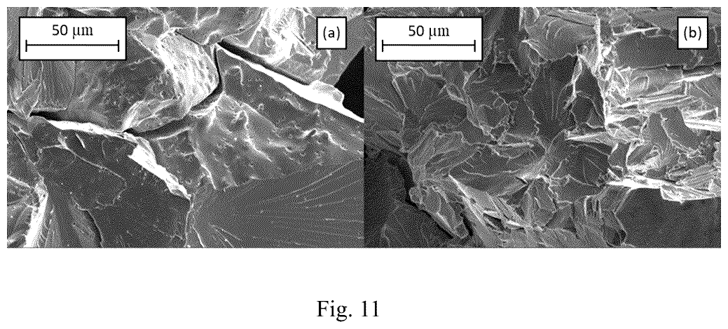

FIG. 11 shows representative fracture surfaces of broken disk bend samples for vanadium-free materials (left; (a)) and vanadium-containing materials (right; (b)). All vanadium-containing materials showed complete transgranular cleavage, as predicted by the model. Vanadium-free materials were expected to be entirely intergranular fracture, but all exhibited mixed mode fracture with significant regions of both intergranular and transgranular cleavage. The regions in vanadium-free materials that exhibited transgranular cleavage are likely due to the relatively large grain size of the material compared to the vanadium-containing materials, along with potential alignment of cleaved grains to dislocation motion and strain fields.

### 2. Discussion

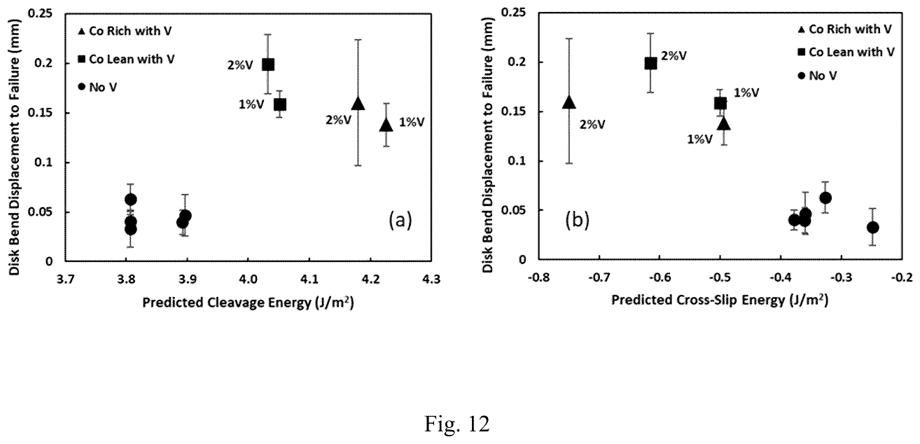

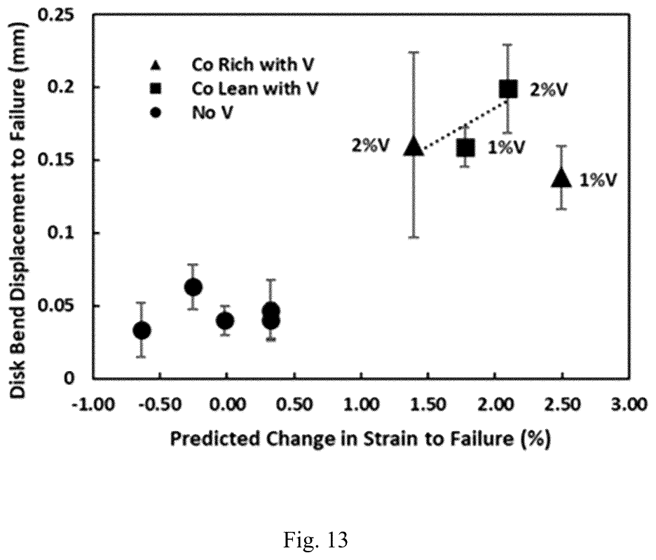

The relationships between predicted failure strain and measured disk bend results in the vanadium-containing cobalt-lean and cobalt-rich materials (FIG. 10) demonstrated that the model was sensitive to stoichiometry differences in alloys. The two primary features of the model that were sensitive to stoichiometry were the cleavage energies and the impact of cross-slip energy to strain accumulation. FIGS. 12 and 13 show the relationships of these two features to the collected disk bend results. In FIG. 12, intergranular cleavage energy is shown for vanadium-free alloys while transgranular cleavage energy is predicted for vanadium-containing alloys.

Because the basis of the model is energetic competition between the accommodation of plastic strain and cleavage, higher cleavage energy is predicted to directly increase cleavage strain. Disk bend results showed that higher predicted cleavage energies correlate with lower failure strain along with separate groups of cobalt-lean and cobalt-rich materials similar to those observed in overall failure strain predictions (FIG. 12(a)). This could indicate that transgranular fracture energy does not change significantly as a function of chemistry, or that vanadium has an impact on transgranular cleavage energy, but stoichiometry of iron and cobalt has minimal impact.

FIG. 12(b) shows that the 2 at. % vanadium cobalt-rich alloy is predicted to have a significantly more negative cross-slip energy, and therefore greater dislocation accommodation and higher strain to failure, than all other vanadium-containing alloys. However, this alloy showed lower disk bend performance than the 2 at. % vanadium cobalt-lean alloy. The 2 at. % vanadium cobalt-rich alloy is the only alloy for which vanadium substitutions would be expected to occur primarily on cobalt sites. Since thermodynamic stability of secondary phases was not incorporated in the model, an investigation of the potential impact of secondary phases was performed. Table 5 shows the results of Thermo-Calc software equilibrium models performed using the TCFE Steels/Fe-alloys database version 10.1 at 300K. (Thermo-Calc Software TCFE Steels/Fe-Alloys database version 10.1 (accessed 15 Sep. 2022)). These results predicted that the Co3V phase is present in all but one vanadium-containing alloy, which would have the effect of both reducing the vanadium in solution in the B2 phase and altering the stoichiometry of the B2 phase to be richer in iron, thus changing the model predictions.

TABLE 5
Thermodynamic predictions of the amount of Co₃V
formed at 300K and the resulting B2 chemistry after
precipitation for vanadium-containing alloys.

| | | | | Co₃V | | | |
|---|---|---|---|---|---|---|---|
| | Overall Chemistry (%) | | | Formed | B2 Chemistry (%) | | |
| Alloy | V | Fe | Co | (wt. %) | V | Fe | Co |
| 2 at. % V, | 2.09 | 48.40 | 49.51 | 2.43 | 1.52 | 49.61 | 48.87 |
| Co-rich | | | | | | | |
| 1 at. % V, | 1.08 | 49.64 | 49.28 | 0.24 | 1.02 | 49.76 | 49.22 |
| Co-rich | | | | | | | |
| 1 at. % V, | 1.77 | 50.05 | 48.18 | 0.00 | 1.77 | 50.05 | 48.18 |
| Co-lean | | | | | | | |
| 2 at. % V, | 2.22 | 49.46 | 48.31 | 0.34 | 2.16 | 49.62 | 48.22 |
| Co-lean | | | | | | | |

FIG. 13 shows the strain to failure predictions using the corrected B2 chemistries from Table 5. While the 2 at. % vanadium cobalt-rich alloy prediction better aligned with the cobalt-lean chemistries using the corrected B2 chemistries, the 1 at. % vanadium cobalt-rich alloy still had a higher predicted ductility than measured disk bend failure displacement.

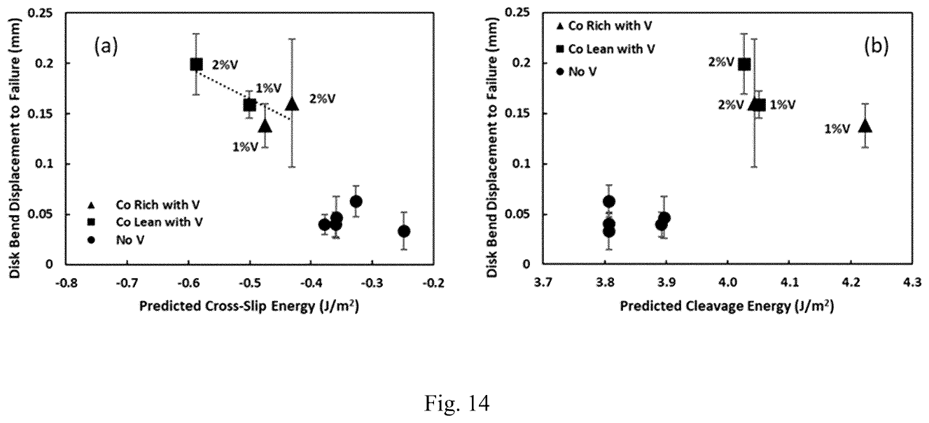

In FIG. 14, intergranular cleavage energy is predicted for vanadium-free alloys while transgranular cleavage energy is predicted for vanadium-containing alloys. FIG. 14(a) shows that, with the corrected B2 chemistries, the expected relationship of increasing disk bend failure displacement with decreasing cross-slip energy was observed. This trend does not intersect the grouping of vanadium-free alloys, which suggests that there is some step function that occurs when cross-slip energy is reduced sufficiently to enable higher levels of ductility, likely through the predicted and observed shift to transgranular cleavage.

FIG. 14(b) shows the updated cleavage energy predictions. The 1 at. % vanadium cobalt-rich alloy had significantly higher predicted cleavage energy than all other vanadium-containing alloys. The linear model for transgranular cleavage is based on only one amount of vanadium addition to the alloy, which don't match the chemistry of any of the arc melted materials. This disparity between the model and experimental results suggests that a crack tip blunting effect, which is not accounted for in the model, or a deviation from linearity in cleavage energies as a function of vanadium content is what is causing the observed cleavage energy trend and the model inaccuracies.

### 3. Cleavage Mode Protection

The previously developed criteria for predicting the change in cleavage mode from intergranular to transgranular cleavage with the addition of vanadium incorporates the theory that dislocations build up on grain boundaries and enable easier transmission of strain across the grain boundaries. While the experimental results all fail with the predicted cleavage mode using this criteria, the minimum amount of vanadium predicted to drive a shift in cleavage mode was not identified due to experimental resolution.

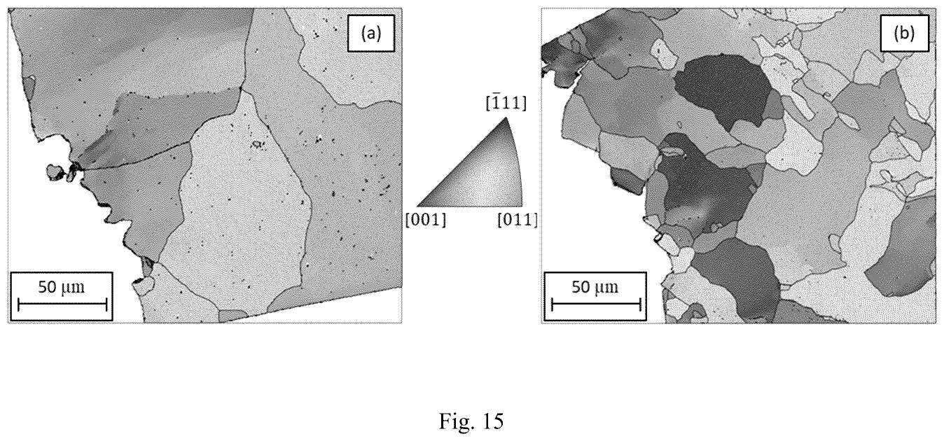

To provide validation for the theory of strain transmission across grain boundaries, EBSD scans of broken disks adjacent to failure surfaces were performed. FIG. 15 shows inverse pole figures for the equiatomic alloy (left; (a)) and the 2 at. % vanadium cobalt-rich alloy (right; (b)). The grain size of the equiatomic alloy was significantly larger than the vanadium-containing alloy; this trend was present in all of the evaluated materials. While grain size might have some impact on ductility, the impact is minor and has only been suggested to occur in vanadium- or nickel-containing Fe—Co alloys. (Sourmail, Near Equiatomic FeCo Alloys: Constitution, Mechanical and Magnetic properties. Progress in Materials Science, 50(7), 816-880 (2005).) In addition, the larger the grains, the lower the probability that grains are oriented to easily enable strain transmission across grain boundaries. While disk bend testing literature recommends at least ten grains through the thickness of a sample to avoid this issue, the relative error in ductility of the large-grained samples was reasonably small, even when only a few grains spanned the thickness of disks.

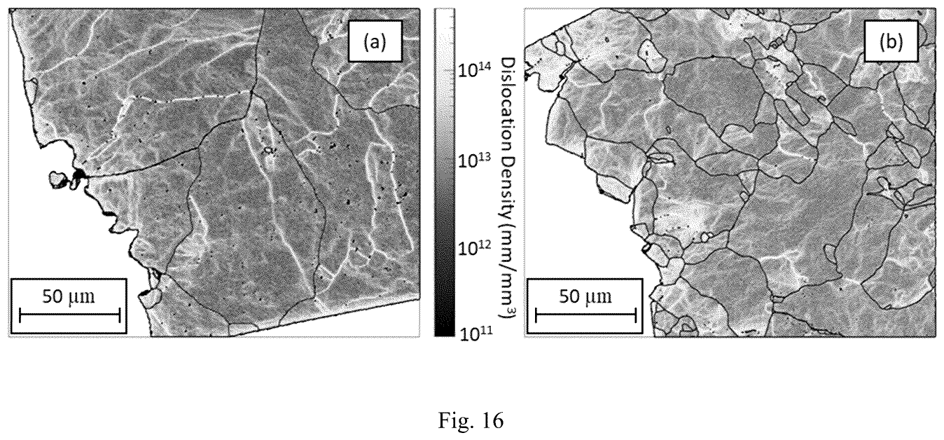

The data collected from the EBSD scans was evaluated for dislocation density using MTEX algorithms. (Pantleon, Resolving the geometrically necessary dislocation content by conventional electron backscattering diffraction. Scripta Materialia, 58(11), 994-997 (2008).) Results are shown in FIG. 16. Overall, the dislocation density values calculated from the EBSD data were similar to the predicted dislocation densities for failure strain. The equiatomic alloy and the 2 at. % vanadium cobalt-rich alloy had average failure dislocation densities of approximately 1E13 m/m³ and 5E13 m/m³, respectively. Dislocation bands are evident in each image as regions of higher dislocation density than the lower density background regions. Previous work has indicated that ordered Fe—Co alloys have planar dislocation glide, and disordered Fe—Co alloys have wavy glide. (Stoloff et al., The plastic Deformation of Ordered FeCo and Fe³ Al alloys. Acta Metallurgica, 12(5), 473-485 (1964).) However, in this study, ordered samples both with and without vanadium showed wavy glide.

This observation of wavy glide in all cases is supported by the simulation work done to develop the present model, which indicated the ability to cross-slip in ordered material, as well as Yamguchi et. al. (Slip Systems in an Fe-54% Co Alloy. Scripta Mater., 16:607-609 (1982)), who showed deformation of a single crystal of Fe—Co. Also observed in the dislocation density maps were differences in the population of dislocation bands, which transmitted strain across grain boundaries. The ordered equiatomic alloy had far fewer occurrences of strain transmission than the ordered vanadium-containing alloy. FIG. 16(a) shows several examples of dislocation bands that terminated at grain boundaries and only a few cases where strain transmission occurred. FIG. 16(b) shows many cases of dislocations that transitioned across grain boundaries.

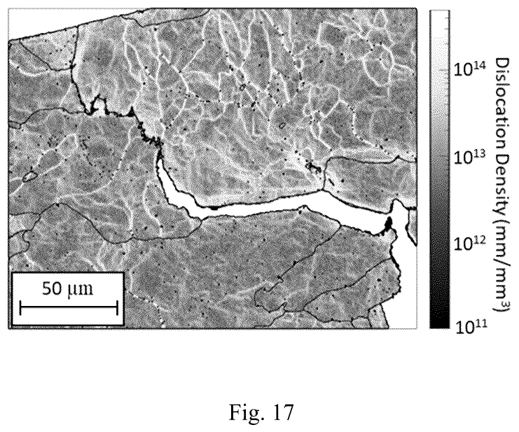

There is a statistical component to the ability of strain to transmit across grain boundaries. If two adjacent crystals are aligned such that the angles between their respective slip planes are small, it is more likely that strain can transmit. FIG. 17 shows an example of a single disk bend sample that had no vanadium but exhibited significantly higher displacement to failure than all other samples of the same chemistry. The background dislocation density in this material was similar to that of the equiatomic alloy (FIG. 16(a)). However, there was an increase in the population of dislocation bands in the upper grain and an increased population of dislocations that transmitted across grain boundaries, even in the region of the crack. Observations of higher strain transmission across grain boundaries when crystal orientations are well-aligned confirms the orientation relationship sensitivity of the transition from intergranular to transgranular fracture.

The observation of a higher occurrence of strain transmission across grain boundaries in vanadium-containing materials despite minimal changes in predicted cleavage energy shows that the improvement of cross-slip energies, which enables transmission of strain across grain boundaries and thus drives a change in cleavage mode to transgranular, is the primary cause of improved ductility in Fe—Co alloys with the addition of vanadium. This invalidates the theory that Fe—Co ductility is a function of changes in cleavage energy, like those observed in Ni₃Al alloys with boron additions. Bond et al., Effect of Boron on the Mechanism of Strain Transfer Across Grain Boundaries in Ni₃Al, Journal of Materials Research, 2(4), 436-440 (1987).

In conclusion, these results showed that the model correctly predicted the major trends with vanadium additions. The developed model, along with the disk bend results and dislocation density maps, strongly indicates that the primary mechanism for the increase in ductility of Fe—Co alloys with vanadium additions is an increase in strain transmission across grain boundaries, which is enabled by reductions in cross-slip energies through the manipulation of APB energies. The transmission of strain across grain boundaries directly drives the observed shift from intergranular cleavage to transgranular cleavage.

1. A method of forming a final alloy from a base alloy comprising a first quantity of a first metal and a first quantity of a chemical element different from said first metal, said method comprising determining whether a predetermined difference between the strain accommodation energy of said base alloy and the cleavage energy of said base alloy would exist, wherein:
(I) if said predetermined difference would exist, forming said final alloy by subjecting said respective first quantities to an alloy formation process; or
(II) if said predetermined difference would not exist, determining whether a modified base alloy would achieve said predetermined difference, wherein said modified base alloy would comprise a modification chosen from:
(a) a second quantity of said first metal different from said first quantity of said first metal;
(b) a second quantity of said chemical element different from said first quantity of said chemical element;
(c) a ductility component different from said first metal and different from said chemical element, wherein said ductility component would either be absent from said base alloy or would be present in said base alloy in a quantity different from that in said modified base alloy;
(d) two of (a), (b), or (c); or
(e) each of (a), (b), and (c), and
if said modified base alloy would achieve said predetermined difference, forming the final alloy by subjecting said first metal, said chemical element, and any ductility component to an alloy formation process; or
if said modified base alloy would not achieve said predetermined difference:
repeating this (II) with one or more further modified base alloys until a further modified base alloy is identified that would achieve said predetermined difference, said further modified base alloy having respective final quantities of said first metal, said chemical element, and any ductility component that may be present; and
forming the final alloy by subjecting said respective final quantities to an alloy formation process.

2. The method of claim 1, wherein said base alloy comprises said ductility component in a first quantity, and (II)(c) comprises said ductility component present in said base alloy in a quantity different from that in said modified base alloy.

3. The method of claim 1, wherein the final alloy comprises:
(i) less of said ductility component than of said first metal;
(ii) less of said ductility component than of said chemical element different from said first metal; or
(iii) both (i) and (ii).

4. The method of claim 1, wherein said first metal is chosen from cobalt, iron, lithium, sodium, potassium, rubidium, cesium, francium, beryllium, magnesium, calcium, strontium, barium, radium, scandium, yttrium, titanium, zirconium, hafnium, rutherfordium, vanadium, niobium, tantalum, dubnium, chromium, molybdenum, tungsten, seaborgium, manganese, technetium, rhenium, bohrium, ruthenium, osmium, hassium, rhodium, iridium, meitnerium, nickel, palladium, platinum, darmstadtium, copper, silver, gold, roentgenium, zinc, cadmium, mercury, copernicium, aluminum, gallium, indium, thallium, nihonium, tin, lead, flerovium, bismuth, moscovium, polonium, livermorium, tennessine, or mixtures thereof.

5. The method of claim 1, wherein said chemical element different from said first metal is chosen from metals, metalloids, interstitial elements, or combinations thereof.

6. The method of claim 1, wherein said chemical element different from said first metal is chosen from cobalt, iron, lithium, sodium, potassium, rubidium, cesium, francium, beryllium, magnesium, calcium, strontium, barium, radium, scandium, yttrium, titanium, zirconium, hafnium, rutherfordium, vanadium, niobium, tantalum, dubnium, chromium, molybdenum, tungsten, seaborgium, manganese, technetium, rhenium, bohrium, ruthenium, osmium, hassium, rhodium, iridium, meitnerium, nickel, palladium, platinum, darmstadtium, copper, silver, gold, roentgenium, zinc, cadmium, mercury, copernicium, aluminum, gallium, indium, thallium, nihonium, tin, lead, flerovium, bismuth, moscovium, polonium, livermorium, tennessine, boron, silicon, germanium, arsenic, antimony, tellurium, astatine, carbon, oxygen, sulfur, phosphorus, or mixtures thereof.

7. The method of claim 1, wherein said ductility component is chosen from metals, metalloids, interstitial elements, or combinations thereof.

8. The method of claim 1, wherein the first quantity of said first metal is within about 20 at. % of the first quantity of said chemical element different from said first metal.

9. The method of claim 1, wherein said first metal is cobalt, and said chemical element is iron.

10. The method of claim 1, wherein said ductility component is chosen from vanadium, nickel, niobium, titanium, chromium, molybdenum, tungsten, manganese, copper, zinc, cobalt, lithium, sodium, potassium, rubidium, cesium, francium, beryllium, magnesium, calcium, strontium, barium, radium, scandium, yttrium, zirconium, hafnium, rutherfordium, vanadium, tantalum, dubnium, seaborgium, technetium, rhenium, bohrium, ruthenium, osmium, hassium, rhodium, iridium, meitnerium, palladium, platinum, darmstadtium, silver, gold, roentgenium, cadmium, mercury, copernicium, aluminum, gallium, indium, thallium, nihonium, tin, lead, flerovium, bismuth, moscovium, polonium, livermorium, tennessine, boron, silicon, germanium, arsenic, antimony, tellurium, astatine, carbon, oxygen, sulfur, phosphorus, or mixtures thereof.

11. The method of claim 1, wherein said first metal is cobalt, said chemical element different from said first metal is iron, and said ductility component is vanadium.

12. The method of claim 1, wherein said ductility component is present in the final alloy at a level of less than about 20 at. %.

13. The method of claim 1, wherein the atomic ratio of said first metal to said chemical element in the final alloy is about 0.8 to about 1.2.

14. The method of claim 1, wherein said predetermined difference comprises the cleavage energy being greater than the strain accommodation energy.

15. The method of claim 1, wherein said alloying process is chosen from one or more of powder metallurgy processes, casting processes, or melt atomization processes.

16. The method of claim 1, wherein said base alloy comprises an additional component different from said first metal and different from said chemical element.

17. The method of claim 16, wherein said additional component is chosen from metals, metalloids, interstitial elements, or combinations thereof.

18. The method of claim 16, wherein said additional component is chosen from cobalt, lithium, sodium, potassium, rubidium, cesium, francium, beryllium, magnesium, calcium, strontium, barium, radium, scandium, yttrium, titanium, zirconium, hafnium, rutherfordium, vanadium, niobium, tantalum, dubnium, chromium, molybdenum, tungsten, seaborgium, manganese, technetium, rhenium, bohrium, ruthenium, osmium, hassium, rhodium, iridium, meitnerium, nickel, palladium, platinum, darmstadtium, copper, silver, gold, roentgenium, zinc, cadmium, mercury, copernicium, aluminum, gallium, indium, thallium, nihonium, tin, lead, flerovium, bismuth, moscovium, polonium, livermorium, tennessine, boron, silicon, germanium, arsenic, antimony, tellurium, astatine, carbon, oxygen, sulfur, phosphorus, or combinations thereof.

19. An alloy formed according to the method of claim 1.

20. An alloy formed according to the method of claim 14.
